(19) United States
(12) Patent Application Publication
(10) Pub. No.: US 2021/0196673 A1
(43) Pub. Date: Jul. 1, 2021

(54) COMPOSITIONS AND METHODS FOR TREATING AND PREVENTING RESPIRATORY VIRAL INFECTIONS USING GREEN TREE EXTRACT

(71) Applicant: Augusta University Research Institute, Inc., Augusta, GA (US)

(72) Inventor: Stephen D. Hsu, Evans, GA (US)

(73) Assignee: Augusta University Research Institute, Inc., Augusta, GA (US)

(21) Appl. No.: 17/182,425

(22) Filed: Feb. 23, 2021

Related U.S. Application Data
(63) Continuation-in-part of application No. 16/541,247, filed on Aug. 15, 2019.
(60) Provisional application No. 62/764,974, filed on Aug. 17, 2018.

(51) Int. Cl. A61K 31/353; A61K 47/10; A61K 9/00; A61P 31/14; A61P 31/16

(52) U.S. Cl. CPC A61K 31/353; A61K 47/10; A61P 31/16; A61K 9/0014; A61P 31/14; A61K 9/0073

(57) ABSTRACT

Modified green tea polyphenol compositions and their methods of use in treating and preventing SARS-CoV-2 infections are provided. An exemplary green tea polyphenol composition includes (−)-epigallocatechin-3-gallate that can be esterified with a C₁-C₃₀ group in at least one position and a carrier such as glycerol. The modified green tea polyphenol compositions can be used to treat and prevent SARS-CoV-2 infections without coming into contact with the viral cell.

### CROSS-REFERENCE TO RELATED APPLICATIONS

This application is a continuation-in-part application of U.S. patent application Ser. No. 16/541,247 filed on Aug. 15, 2019, and claims benefit of and priority to U.S. Provisional Patent Application No. 62/764,974, filed on Aug. 17, 2018, all of which are incorporated by reference in their entirety.

### STATEMENT REGARDING FEDERALLY SPONSORED RESEARCH

This invention was made with government support under R42AI124738 awarded by the National Institutes of Health and the non-clinical services program offered by the National Institute of Allergy and Infectious Diseases.

### TECHNICAL FIELD OF THE INVENTION

The invention is generally directed to compositions and methods of use thereof for the treatment, and prevention of SARS-CoV-2 infections.

### BACKGROUND OF THE INVENTION

The novel coronavirus, SARS-CoV-2, has caused a global pandemic of COVID-19 respiratory disease, with millions infected, hundreds of thousands of deaths, and many survivors dealing with multiple long-term adverse health effects. The burdens on healthcare and the economy have not been seen since the 1918 flu pandemic, which was caused by another respiratory virus. Therefore, therapeutic and prophylactic methods against SARS-CoV-2 are in urgent need. While certain antiviral drugs, such as Remdesivir, provide relief to certain patient populations, many other existing antiviral drugs or combinations of pharmaceuticals have yet to show clinical efficacy against COVID-19. Compounds that possess strong virucidal properties with different mechanisms of action against a broad-spectrum of viruses may provide novel approaches to combat SARS-CoV-2, especially if the compounds are classified as generally considered as safe (GRAS) by the FDA.

COVID-19 is a viral disease that affects the epithelial cells of the respiratory system and causes inflammation of the mucosal membrane. This leads to alveolar damage and eventually pneumonia. It is caused by severe acute respiratory syndrome corona virus-2 (SARS-CoV-2), commonly known as corona virus, a positive-sense of single-stranded RNA virus. Before SARS-CoV-2, two more epidemic diseases were caused by corona viruses, namely, severe acute respiratory syndrome (SARS) and middle eastern respiratory syndrome (MERS). The mortality rate due to SARS or MERS are in hundreds. On the contrary, the mortality rate due to SARS-CoV-2 is exceptionally high. Till todate, no drug molecules or specific therapies have been developed to combat COVID-19. Considering the risk factors associated with this viral infection, an effective drug molecule is urgently required for effective treatment and to limit the transmission of this disease.

Green tea polyphenols are known phytochemicals that have antioxidant, anti-microbial, anticancer and anti-inflammatory properties. Extracts from green tea have been used for medicinal purposes for generations in China. Green tea polyphenol in extracts are mostly water-soluble and can be easily oxidized if they are mixed in emulsions containing water, such as detergents and cosmetics.

Therefore, it is very plausible to investigate whether green tea polyphenols can be considered as therapeutic agents to treat severe viral respiratory infections including current COVID-19.

Therefore, there is an urgent need for more effective, innocuous treatments and preventative measures for SARS-CoV-2 infection,

Therefore, it is an object of the invention to provide compositions and methods of use thereof for treating and preventing SARS-CoV-2 infections.

It is also an object of the invention to provide compositions and methods of use thereof for inhibiting or reducing SARS-CoV-2 infections.

It is a further object of the invention to provide compositions and methods of use thereof for preventing the replication SARS-CoV-2.

### SUMMARY OF THE INVENTION

Methods and compositions for inhibiting or reducing viral infections are provided. In one aspect, the invention provides a method for inhibiting or reducing respiratory viral infection in a subject by administering to the subject an effective amount of a composition comprising at least one green tea polyphenol esterified with a C₁-C₃₀ group in at least one position and a carrier to inhibit or reduce entry of SARS-CoV-2 into respiratory epithelial cells of the subject.

In some aspects, the carrier is either in water soluble form or in lipid soluble form.

One embodiment provides a method of reducing the risk of a viral infection in as subject by administering to the subject an effective amount of a prophylactic composition comprising (−)-epigallocatechin-3-gallate-palmitate and glycerol to inhibit or reduce viral infection in respiratory epithelial cells of the subject.

Another embodiment provides a method for preventing the replication of respiratory viral infection in a subject by administering to the subject an effective amount of a composition containing at least one green tea polyphenol esterified with a C₁-C₃₀ group in at least one position and a carrier to prevent entry of SARS-CoV-2 into respiratory epithelial cells of the subject.

Still another embodiment provides a composition containing a prophylactically effective amount of epigallocatechin-3-gallate-palmitate and glycerol to inhibit or reduce viral entry into respiratory epithelial cells of a subject, wherein the composition is formulated for nasal, bronchial, or pulmonary administration.

One embodiment provides a method for inhibiting or reducing a viral infection in a subject by administering to the subject a composition containing an effective amount of a green tea polyphenol esterified with a C₁-C₃₀ group in at least one position and a carrier to inhibit or reduce entry of the virus into respiratory epithelial cells of the subject. The green tea polyphenol can be (−)-epicatechin, (−)-epigallocatechin, (−)-epicatechin-3-gallate, or a proanthocyanidin. In one embodiment the esterified green tea polyphenol is (−)-epigallocatechin-3-gallate-palmitate. In one embodiment, the carrier is glycerol.

In some embodiments, the virus is a respiratory virus. In other embodiments, the virus is an influenza virus, respiratory syncytial virus, parainfluenza virus, adenovirus, rhinovirus, or a coronavirus. In some embodiments, the virus is a SARS-CoV-2.

The esterified green tea polyphenol can be formulated into a pharmaceutical composition. For example, the esterified green tea polyphenol can be formulated for delivery into the upper respiratory system. Exemplary formulations include nasal, bronchial, oral, inhalational, and pulmonary formulations. In some embodiments, the modified green tea polyphenol is formulated for topical administration including but not limited to a liquid, gel, wax, vaper, or paste. In other embodiments, the composition is formulated as an aerosol. The aerosol can be a liquid or powdered aerosol. In some embodiments, the composition contains one or more pharmaceutically acceptable excipients such as glycerin. The composition can contain 0.01%-20% w/v of the esterified green tea polyphenol and 10% to 20% glycerol.

It has been discovered that esterified green tea polyphenols can coat the surface of cells, in particular, respiratory epithelial cells and block, inhibit, or reduce viral uptake in the coated cells. Exemplary respiratory epithelial cells include but are not limited to ciliated cells, goblet cells, basal cells, epidermal cells, and combinations thereof. The respiratory epithelial cells are typically at least partially coated with the esterified green tea polyphenol compounds to block or inhibit virus uptake by the coated cells.

### BRIEF DESCRIPTION OF THE DRAWINGS

FIG. 1 is a graph showing the results of a viral inactivation test of H1N1 exposure by direct contact with EC16. EC16 was tested at two concentrations (0.01% and 0.1%) in two concentrations of carrier (10% and 20%) by mixing with H1N1 virus for 1 minute before MDCK infection and TCID50 determination. Three replicate experiments were performed using 0.01% and 0.1% EC16 in 10% carrier and 0.1% EC16 in 20% carrier; 0.01% EC16 in 20% carrier was tested separately, and three additional repeat tests (open symbols) were performed for 0.1% EC16 in 10% carrier. Mean logit transformed percentage values (large horizontal bar) and standard deviations (small horizontal bars) are shown.

FIG. 2 is a graph showing the effect of EC16 pre-treatment of MDCK cells on viral titer (prevention effect). MDCK cell monolayers were incubated with EC16 treatments for 1 hour, free EC16 was then washed away, and cells exposed to virus for one hour followed by TCID50 assay. Combined data from two sets of experiments are shown, one comparing 0.1% EC16 at different carrier concentrations (solid symbols) and one comparing different EC16 concentrations at 20% carrier. Mean logit transformed percentage values (large horizontal bar) and standard deviations (small horizontal bars) are shown for combined data.

FIG. 3 is a bar graph showing the duration of the EC16 preventative effect on H1N1 infection in cells pre-treated with EC16 before H1N1 infection. The cells were treated with 0.1% EC16, 0.05% EC16, DMSO plus EC16, or control for 1 hour, the EC16 was washed away and the cells were incubated in media for an hour without EC16, then the cells were infected with H1N1. The X-axis represents treatment group and the Y-axis represents relative percent infection.

FIG. 4 is a graph showing the effect of EC16 formulations on viral reproduction in MDCK cells infected with virus (treatment effect). Monolayers of MDCK cells were infected with a series of dilutions of H1N1 for 1 hour, then EC16 formulations were applied for 1 hour prior to TCID50 assay. The X-axis represents EC16 formulation (carrier % and EC16%) and the Y-axis represents infectivity (logit %). Mean logit transformed percentage values (large horizontal bar) and standard deviations (small horizontal bars) are shown.

FIG. 5 is a graph showing the effect of a thin layer of EC16 formulation coating MDCK cells on subsequent H1N1 viral infection (thin layer prevention effect). The X-axis represents experimental parameters (time of EC16 treatment (Time (mins)) and EC16 formulation (carrier % and EC16%) and the Y-axis represents infectivity (logit %). Mean logit transformed percentage values (large horizontal bar) and standard deviations (small horizontal bars) are shown.

FIG. 6 is a bar graph showing formulation cytotoxic effects by MTT assay. The X-axis represents the EC16 formulation (carrier % and EC16%) and the Y-axis represents optical density (OD). Mean values (n=16) and standard deviations (error bars) are shown.

### DETAILED DESCRIPTION OF THE INVENTION

### I. Definitions

The following terms are intended to have the meanings presented therewith below and are useful in understanding the description and intended scope of the present invention.

When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term ‘substituted’ is to be defined as set out below. It should be further understood that the terms “groups” and “radicals” can be considered interchangeable when used herein.

The articles “a” and “an” may be used herein to refer to one or to more than one (i.e., at least one) of the grammatical objects of the article. By way of example “an analogue” means one analogue or more than one analogue.

As used herein, the term “pharmaceutical composition” means a mixture comprising a pharmaceutically acceptable active ingredient, in combination with suitable pharmaceutically acceptable excipients. In one embodiment the pharmaceutically acceptable ingredient is a pharmaceutically acceptable acid addition salt of the compound of formula I, or a solvate or hydrate of this acid addition salt.

Pharmaceutical excipients are substances other than the pharmaceutically acceptable active ingredient which have been appropriately evaluated for safety and which are intentionally included in an oral solid dosage form. For example, excipients can aid in the processing of the drug delivery system during its manufacture, protect, support or enhance stability, bioavailability or patient acceptability, assist in product identification, or enhance any other attribute of the overall safety, effectiveness or delivery of the drug during storage or use. Examples of excipients include, for example but without limitation inert solid diluents (bulking agent e.g., lactose), binders (e.g., starch), glidants (e.g., colloidal silica), lubricants (e.g., non-ionic lubricants such as vegetable oils), disintegrants (e.g., starch, polivinylpyrrolidone), coating better polymers (e.g., hydroxypropyl methylcellulose), colorants (e.g., iron oxide), and/or surfactants (e.g., non-ionic surfactants).

As used herein, the term “pharmaceutical formulation” means a composition in which different chemical substances, including the active drug, are combined to produce a final medicinal product. Examples of formulation include enteral formulations (tablets, capsules), parenteral formulations (liquids, lyophilized powders), or topical formulations (cutaneous, inhalable).

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of formula I or derivatives thereof that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g. an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. More particularly, such salts are formed with hydrobromic acid, hydrochloric acid, sulfuric acid, toluenesulfonic acid, benzenesulfonic acid, oxalic acid, maleic acid, naphthalene-2-sulfonic acid, naphthalene-1,5-disulfonic acid, 1-2-ethane disulfonic acid, methanesulfonic acid, 2-hydroxy ethanesulfonic acid, phosphoric acid, ethane sulfonic acid, malonic acid, 2-5-dihydroxybenzoic acid, or L-Tartaric acid.

The term “pharmaceutically acceptable cation” refers to an acceptable cationic counter-ion of an acidic functional group. Such cations are exemplified by sodium, potassium, calcium, magnesium, ammonium, tetraalkylammonium cations, and the like.

“Pharmaceutically acceptable vehicle” refers to a diluent, adjuvant, excipient or carrier with which a compound of the invention is administered.

“Solvate” refers to forms of the compound that are associated with a solvent, usually by a solvolysis reaction. This physical association includes hydrogen bonding. Conventional solvents include water, ethanol, acetic acid and the like. The compounds of the invention may be prepared e.g. in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. ‘Solvate’ encompasses both solution-phase and isolable solvates. Representative solvates include hydrates, ethanolates and methanolates.

The terms “inert solid diluent” or “solid diluent” or “diluents” refer to materials used to produce appropriate dosage form size, performance and processing properties for tablets and/or capsules. An inert solid diluent can be also referred to as filler or filler material. Particular examples of diluents include cellulose powdered, silicified microcrystalline cellulose acetate, compressible sugar, confectioner's sugar, corn starch and pregelatinized starch, dextrates, dextrin, dextrose, erythritol, ethylcellulose, fructose, fumaric acid, glyceryl palmitostearate, inhalation lactose, isomalt, kaolin, lactitol, lactose, anhydrous, monohydrate and corn starch, spray dried monohydrate and microcrystalline cellulose, maltodextrin, maltose, mannitol, medium-chain triglycerides, microcrystalline cellulose, polydextrose, polymethacrylates, simethicone, sorbitol, pregelatinized starch, sterilizable maize, sucrose, sugar spheres, sulfobutylether β-cyclodextrin, talc, tragacanth, trehalose, or xylitol. More particular examples of diluents include cellulose powdered, silicified microcrystalline cellulose acetate, compressible sugar, corn starch and pregelatinized starch, dextrose, fructose, glyceryl palmitostearate, anhydrous, monohydrate and corn starch, spray dried monohydrate and microcrystalline cellulose, maltodextrin, maltose, mannitol, medium-chain triglycerides, microcrystalline cellulose, polydextrose, sorbitol, starch, pregelatinized, sucrose, sugar spheres, trehalose, or xylitol.

“Lubricant” refers to materials that prevent or reduce ingredients from clumping together and from sticking to the tablet punches or capsule filling machine. Lubricants also ensure that tablet formation and ejection can occur with low friction between the solid and die wall. Particular examples of lubricants include canola oil, hydrogenated castor oil, cottonseed oil, glyceryl behenate, glyceryl monostearate, glyceryl palmitostearate, medium-chain triglycerides, mineral oil, light mineral oil, octyldodecanol, poloxamer, polyethylene glycol, polyoxyethylene stearates, polyvinyl alcohol, starch, or hydrogenated vegetable oil. More particular examples of diluents include glyceryl behenate, glyceryl monostearate, or hydrogenated vegetable oil.

“Disintegrant” refers to material that dissolve when wet causing the tablet to break apart in the digestive tract, releasing the active ingredients for absorption. They ensure that when the tablet is in contact with water, it rapidly breaks down into smaller fragments, facilitating dissolution. Particular examples of disintegrants include alginic acid, powdered cellulose, chitosan, colloidal silicon dioxide, corn starch and pregelatinized starch, crospovidone, glycine, guar gum, low-substituted hydroxypropyl cellulose, methylcellulose, microcrystalline cellulose, or povidone.

The term “colorant” describes an agent that imparts color to a formulation. Particular examples of colorants include iron oxide, or synthetic organic dyes (US Food and Drug administration, Code of Federal Regulations, Title 21 CFR Part73, Subpart B).

The term “plasticizing agent” or “plasticizer” refers to an agent that is added to promote flexibility of films or coatings. Particular examples of plasticizing agent include polyethylene glycols or propylene glycol.

The term “pigment” in the context of the present invention refers to an insoluble coloring agent.

The term “film-coating agent’ or ‘coating agent’ or ‘coating material’ refers to an agent that is used to produce a cosmetic or functional layer on the outer surface of a dosage form. Particular examples of film-coating agent include glucose syrup, maltodextrin, alginates, or carrageenan.

“Glidant” refers to materials that are used to promote powder flow by reducing interparticle friction and cohesion. These are used in combination with lubricants as they have no ability to reduce die wall friction. Particular examples of glidants include powdered cellulose, colloidal silicon dioxide, hydrophobic colloidal silica, silicon dioxide, or talc. More particular examples of glidants include colloidal silicon dioxide, hydrophobic colloidal silica, silicon dioxide, or talc.

“Flavoring agents” refers to material that can be used to mask unpleasant tasting active ingredients and improve the acceptance that the patient will complete a course of medication. Flavorings may be natural (e.g., fruit extract) or artificial. Non limiting examples of flavoring agents include mint, cherry, anise, peach, apricot, licorice, raspberry, or vanilla.

The term “Subject” includes mammals such as humans. The terms “human”, “patient” and “subject” are used interchangeably herein.

“Effective amount” means the amount of a compound of the invention that, when administered to a subject for treating a disease, is sufficient to effect such treatment for the disease. The ‘effective amount’ can vary depending on the compound, the disease and its severity, and the age, weight, etc., of the subject to be treated.

“Preventing” or “prevention” refers to a reduction in risk of acquiring or developing a disease or disorder (i.e., causing at least one of the clinical symptoms of the disease not to develop in a subject that may be exposed to a disease-causing agent, or predisposed to the disease in advance of disease onset).

The term “prophylaxis” is related to “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Treating” or “treatment” of any disease or disorder refers, in one embodiment, to ameliorating the disease or disorder (i.e., arresting the disease or reducing the manifestation, extent or severity of at least one of the clinical symptoms thereof). In another embodiment “reating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease. As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radioactive isotopes tritium, i.e., ³H, and carbon-14, i.e. ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, and would be useful in Positron, and 13 Emission Topography (PET) studies for examining substrate receptor occupancy.

All isotopic variants of the compounds provided herein, radioactive or not, are intended to be encompassed within the scope of the invention.

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

The term “alkyl” as used herein, whether used alone or as part of another group, refers to a substituted or unsubstituted aliphatic hydrocarbon chain and includes, but is not limited to, straight and branched chains containing from 1 to 20 carbon atoms, preferably from 2 to 20, from 1 to 10, from 2 to 10, from 1 to 8, from 2 to 8, from 1 to 6, from 2 to 6, from 1 to 4, from 2 to 4, from 1 to 3 carbon atoms, unless explicitly specified otherwise. Illustrative alkyl groups can include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4-dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

The term “alkenyl” as used herein, whether used alone or as part of another group, refers to a substituted or unsubstituted aliphatic hydrocarbon chain and includes, but is not limited to, straight and branched chains having 2 to 8 carbon atoms and containing at least one carbon-carbon double bond.

The term “alkynyl” as used herein, whether used alone or as part of another group, refers to a substituted or unsubstituted aliphatic hydrocarbon chain and includes, but is not limited to, straight and branched chains having 1 to 6 carbon atoms and containing at least one carbon-carbon triple bond.

The term “alkoxy” as used herein, whether used alone or as part of another group, refers to alkyl-O— wherein alkyl is hereinbefore defined.

The term “cycloalkyl” as used herein, whether used alone or as part of another group, refers to a monocyclic, bicyclic, tricyclic, fused, bridged or spiro monovalent saturated hydrocarbon moiety, wherein the carbon atoms are located inside or outside of the ring system. Any suitable ring position of the cycloalkyl moiety may be covalently linked to the defined chemical structures. Illustrative cycloalkyl groups can include, but are not limited to, cyclopropyl, cyclopropylmethyl, cyclobutyl, cyclopentyl, cyclohexyl, cyclohexylmethyl, cyclohexylethyl, cycloheptyl, norbornyl, adamantly, spiro[4,5]decanyl, and homologs, isomers and the alike.

The term “aryl” as used herein, whether used alone or as part of another group, refers to an aromatic carbocyclic ring system having 6 to 30 carbon atoms, preferably 6 to 10 carbon atoms, optionally substituted with 1 to 3 substituents independently selected from halogen, nitro cyano, hydroxy, alkyl, alkenyl, alkoxy, cycloalkyl, amino, alkylamino, dialkylamino, carboxy, alkoxycarbonyl, haloalkyl, and phenyl.

The term “phenyl” as used herein, whether used alone or as part of another group, refers to a substituted or unsubstituted phenyl group.

The term “heteroaryl” as used herein, whether used alone or as part of another group, refers to a 3 to 30 membered aryl heterocyclic ring, which contains from 1 to 4 heteroatoms selected from the group consisting of O, N, Si, P and S atoms in the ring and may be fused with a carbocyclic or heterocyclic ring at any possible position.

The term “heterocycloalkyl” as used herein, whether used alone or as part of another group, refers to a 5 to 7 membered saturated ring containing carbon atoms and from 1 to 2 heteroatoms selected from the group consisting of O, N and S atoms.

The term “halogen or halo” as used herein, refers to fluoro, chloro, bromo or iodo.

The term “haloalkyl” as used herein, whether used alone or as part of another group, refers to an alkyl as hereinbefore defined, independently substituted with 1 to 3, F, C₁, Br or I.

The term “about” as used herein, refers that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments. Additionally, in phrase “about X to Y,” is the same as “about X to about Y,” that is the term “about” modifies both “X” and “Y.”

The term “compound” as used herein, refers to salts, solvates, complexes, isomers, stereoisomers, diastereoisomers, tautomers, and isotopes of the compound or any combination thereof.

The term “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are used in their inclusive, open-ended, and non-limiting sense.

The term “racemic” as used herein refers to a mixture of the (+) and (−) enantiomers of a compound wherein the (+) and (−) enantiomers are present in approximately a 1:1 ratio. The terms “substantially optically pure,” “optically pure,” and “optically pure enantiomers,” as used herein, mean that the composition contains greater than about 90% of a single stereoisomer by weight, preferably greater than about 95% of the desired enantiomer by weight, and more preferably greater than about 99% of the desired enantiomer by weight, based upon the total weight.

The term “enantiomer” refers to a stereoisomer that is a non-superimposable mirror image of each other. A diastereomer is a stereoisomer with two or more stereocenters, and the isomers are not mirror images of each other.

As used herein, the terms “green tea catechins (GTC)” and “green tea polyphenols” can be used interchangeably and refer to polyphenolic compounds from the leaves of Camellia sinesus. GTCs have been reported to have various health benefits against numerous diseases. Green tea polyphenols include but are not limited to (−)-epicatechin, (−)-epigallocatechin, (−)-epicatechin-3-gallate, (−)-epigallocatechin-3-gallate, and proanthocyanidins. Modified green tea polyphenol refers to a green tea polyphenol having one or more hydrocarbon chains, for example C₁-C₃₀.

The term “substituted C₁ to C₃₀” refers to an alkyl, alkenyl, or alkynyl chain of one to thirty carbons wherein one or more carbons are independently substituted with one or more groups including, but not limited to, halogen, hydroxy group, aryl group, heterocyclic group, or alkyl ester. The range C₁ to C₃₀ includes C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ etc. up to C₃₀ as well as ranges falling within C₁ to C₃₀, for example, C₁ to C₂₉, C₂ to C₃₀, C₃ to C₂₈, etc. The range also includes less than C₃₀, less than C₁₉, etc.

As used herein, the terms “treat,” “treating,” “treatment” and “therapeutic use” refer to the elimination, reduction or amelioration of one or more symptoms of a disease or disorder. As used herein, a “therapeutically effective amount” refers to that amount of a therapeutic agent sufficient to mediate a clinically relevant elimination, reduction or amelioration of such symptoms. An effect is clinically relevant if its magnitude is sufficient to impact the health or prognosis of a recipient subject. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease.

As used herein, “coating cells” refers to a thin layer or covering of a substance that is applied to the surface of cells.

As used herein, the term “prophylactic agent” refers to an agent that can be used in the inhibition or prevention of a disorder or disease prior to the detection of any symptoms of such disorder or disease. A “prophylactically effective” amount is the amount of prophylactic agent sufficient to mediate such protection. A prophylactically effective amount may also refer to the amount of the prophylactic agent that provides a prophylactic benefit in the prevention of disease.

As used herein, the terms “individual,” “host,” “subject,” and “patient” are used interchangeably herein, and refer to a mammal, including, but not limited to, humans, rodents, such as mice and rats, and other laboratory animals.

Influenza viruses A, B, C, and D belong to the Orthomyxoviridae family. Influenza viruses are enveloped, single-stranded, negative sense RNA viruses. They are spherical or filamentous in shape, with the spherical forms on the order of 100 nm in diameter and the filamentous forms often in excess of 300 nm in length. The influenza A virion is studded with glycoprotein spikes of HA and NA projecting from a host cell-derived lipid membrane.

The influenza viruses have a segmented genome, with influenza A and B virus genomes having eight negative-sense, single-stranded viral RNA segments and influenza C virus genome having a seven-segment genome. The segmented genome enables antigenic shift, in which an influenza A virus strain acquires the HA segment, and possibly the NA segment as well, from an influenza virus of a different subtype. Pandemic influenza arises when antigenic shift generates a virus to which humans are susceptible but immunologically naïve.

Influenza viruses are largely transmitted through airborne respiratory secretions released when an infected individual coughs or sneezes. It enters through the nose or mouth and settles in the respiratory tract. Influenza virus can infect cells of the respiratory tract. The influenza virus life cycle can be divided into the following stages: entry into the host cell; entry of viral ribonuclease protein (vRNP) into the nucleus; transcription and replication of the viral genome; export of the vRNPs from the nucleus; and assembly and budding at the host cell plasma membrane. The replication cycle of influenza viruses, from the time of entry to the production of new virus, is very quick, with shedding of the first influenza viruses from infected cells occurring after only 6 hours.

As used herein, “H1N1 virus” refers to the subtype of influenza A virus that was the most common cause of human influenza in 2009, and is associated with the 1918 outbreak known as the Spanish Flu.

As used herein, “EC16” refers to EGCG-palmitate which is made of one EGCG molecule linked to palmitic acid molecule (16 carbon fatty acid).

As used herein, “glycerol”, “glycerin”, and “glycerine” can be used interchangeably and refer to a sugar alcohol made up of two polyols. Glycerol is a propane molecule attached to three hydroxyl groups.

### II. Modified Green Tea Polyphenol Compositions and Methods of Use

Disclosed herein are modified green tea polyphenol compositions that can prophylactically and therapeutically treat respiratory viruses without requiring direct contact with the virus itself. One embodiment provides a method for inhibiting or reducing viral infection in a subject by administering to the subject a composition containing an effective amount of a green tea polyphenol esterified with a C₁-C₃₀ group in at least one position to inhibit or reduce entry of the virus into respiratory epithelial cells of the subject. The green tea polyphenol can be (−)-epicatechin, (−)-epigallocatechin, (−)-epicatechin-3-gallate, or a proanthocyanidin. In one embodiment the esterified green tea polyphenol is (−)-epigallocatechin-3-gallate-palmitate. In another embodiment, the modified green tea polyphenol composition includes a carrier, such as a sugar alcohol. In one embodiment, the carrier is glycerol. In one embodiment, the respiratory virus is SARS-CoV-2.

A. Green Tea Catechins

Green tea catechins, preferably one or more green tea catechins modified with one or more hydrocarbon chains having C₁ to C₃₀ groups, as well as compositions having one or more green tea catechins, preferably one or more green tea catechins modified with one or more hydrocarbon chains having C₁ to C₃₀ groups, and combinations thereof are provided. Representative green tea polyphenols include, but are not limited to (−)-epigallocatechin-3-gallate, (−)-epicatechin, (−)-epigallocatechin, and (−)-epicatechin-3-gallate. Preferred modified green tea catechins include modified (−)-epigallocatechin-3-gallate.

A modified green tea catechin, a derivative or a variant of a green tea catechin includes green tea catechins having chemical modifications to increase solubility or bioavailability in a host. In certain embodiments, these chemical modifications include the addition of chemical groups having a charge under physiological conditions. In other embodiments the modifications include the conjugation of the green tea catechin to other biological moieties such as polypeptides, carbohydrates, lipids, or a combination thereof. Preferred modifications include modifications with one or more hydrocarbon chains having C₁ to C₃₀ groups.

Another embodiment provides a composition for the prophylactic or therapeutic treatment of respiratory viruses including one or more green tea catechins, modified green tea catechins, optionally in combination with one or more of a pharmaceutically acceptable carrier, diluent, excipient, filler, or other inert or active agents. In some embodiments, the active ingredient in the composition consists essentially of (−)-epigallocatechin-3-gallate, (−)-epigallocatechin-3-gallate modified with one or more hydrocarbon chains having C₁ to C₃₀ groups, or a combination thereof, a pharmaceutically acceptable salt or prodrug thereof. The active ingredient can be in the form a single optical isomer. Typically, one optical isomer will be present in greater than 85%, 90%, 95%, or 99% by weight compared to the other optical isomer. It will be appreciated that the composition can also include at least one additional active ingredient, for example a second therapeutic. Additional description of the disclosed pharmaceutical compositions is provided below.

Green tea polyphenols have poor solubility in lipid medium. Therefore, lipophilic tea polyphenols are also disclosed for use in lipid-soluble medium. Lipophilic tea polyphenols (LTP or Modified green tea polyphenols) can be prepared by catalytic esterification of a green tea polyphenols (GTP).

Compositions containing green tea catechins modified to increase the permeability of the green tea catechins to skin and cell membranes or increase their solubility in hydrophobic media relative to unmodified green tea catechins are therefore provided. Green tea catechins that can be modified include, but are not limited to (−)-epicatechin (EC), (−)-epigallocatechin (EGC), (−)-epicatechin-3-gallate (ECG), (−)-epigallocatechin-3-gallate (EGCG), proanthocyanidins, enantiomers thereof, epimers thereof, isomers thereof, combinations thereof, and prodrugs thereof. One embodiment provides a green tea catechin having an ester-linked C₁ to C₃₀ hydrocarbon chain, for example a fatty acid, at one or more positions. In one embodiment, the fatty acid is palmitic acid, a 16 carbon fatty acid. Another embodiment provides a green tea catechin having one or more cholesterol groups linked to the catechin. The cholesterol group can be linked for example by an ether linkage directly to the catechin or a C₁ to C₁₀ linker can connect the cholesterol group to the catechin.

Another embodiment provides a green tea catechin compound having one or more acyloxy groups, wherein the acyl group is C₁ to C₃₀. It is believed that the addition of alkyl, alkenyl, or alkynyl chains, for example via fatty acid esterification, to green tea catechins increases the stability of the green tea catechins and increases the solubility of the green tea catechins in hydrophobic media including lipids, fats, soaps, detergents, surfactants or oils compared to unmodified green tea catechins. Green tea catechins having one or more hydrocarbon chains, for example ester-linked C₁ to C₃₀ groups or C₁ to C₃₀ acyloxy groups are believed to be more permeable to skin or cell membranes and thereby enable the ester-linked hydrocarbon chain containing green tea catechin or acyloxy containing green tea catechin to readily enter a cell and have a biological effect on the cell, for example modulating gene expression, compared to unmodified green tea catechins.

One embodiment provides a compound according to Formula I:

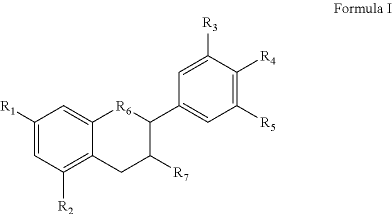

wherein R₁, R₂, R₃, R₄, R₅, and R₇ are each independently H, OH,

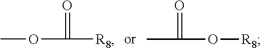

wherein R₈ is a linear, branched or cyclic, saturated or unsaturated, substituted or unsubstituted C₁-C₃₀ group, wherein if R₈ is cyclic, R₈ is a C₃-C₃₀ group; and

R₆ is O, —NR₉R₁₀, or S, wherein R₉ and R₁₀ are independently hydrogen, or a linear, branched, or cyclic, saturated or unsaturated, substituted or unsubstituted C₁-C₃₀ group, wherein if R₉ and/or R₁₀ are cyclic, R₉ and/or R₁₀ are C₃-C₃₀ groups;

wherein at least one of R₁, R₂, R₃, R₄, R₅, R₇, R₉, or R₁₀ is

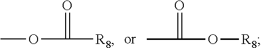

or a pharmaceutically acceptable salt or prodrug thereof, optionally in combination with an excipient.

In preferred embodiments of Formula I, R₈ is a linear or branched alkyl chain. In more preferred embodiments of Formula I, R₈ is a linear or branched C₁₆-C₂₅ alkyl group. In particularly preferred embodiments of Formula I, R₈ is a C₁₇H₃₅ group.

One embodiment provides a compound according to Formula I as described above, provided R₄ is not

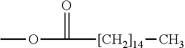

when R₁, R₂, R₃, R₅, and R₇ are OH;
or a pharmaceutically acceptable salt or prodrug thereof, optionally in combination with an excipient.

One embodiment provides a compound according to Formula I as described above wherein at least two of R₁, R₂, R₃, R₄, R₅, or R₇ are independently

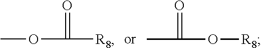

provided R₄ is not

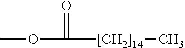

when R₁, R₂, R₃, R₅ are OH, and R₇ is

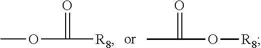

or a pharmaceutically acceptable salt or prodrug thereof, optionally in combination with an excipient.

Another embodiment provides a compound according to Formula I as described above wherein at least three of R₁, R₂, R₃, R₄, R₅, or R₇ are independently

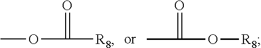

or a pharmaceutically acceptable salt or prodrug thereof, optionally in combination with an excipient.

Still another embodiment provides a compound according to Formula I as described above wherein at least four of R₁, R₂, R₃, R₄, R₅, or R₇ are independently

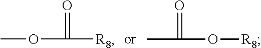

or a pharmaceutically acceptable salt or prodrug thereof, optionally in combination with an excipient.

Another embodiment provides a compound according to Formula II:

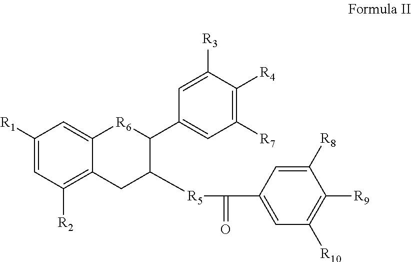

wherein R₁, R₂, R₃, R₄, R₇, R₈, R₉, and R₁₀ are each independently H, OH,

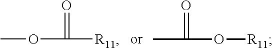

R₁₁ is a linear, branched, or cyclic, saturated or unsaturated, substituted or unsubstituted C₁-C₃₀ group, wherein if R₁₁ is cyclic, R₁₁ is a C₃-C₃₀ group;

R₅ and R₆ are independently O, —NR₁₂R₁₃ or S, wherein R₁₂ and R₁₃ are independently hydrogen, or a linear, branched, or cyclic, saturated or unsaturated, substituted or unsubstituted C₁-C₃₀ group, wherein if Ru and/or R₁₃ are cyclic, Ru and/or R₁₃ are C₃-C₃₀ groups; and

wherein at least one of R₁, R₂, R₃, R₄, R₇, R₈, R₉, and R₁₀ are independently

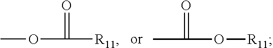

or a pharmaceutically acceptable salt or prodrug thereof, optionally in combination with an excipient.

In preferred embodiments of Formula II, R₁₁ is a linear or branched alkyl chain. In more preferred embodiments of Formula II, R₁₁ is a linear or branched C₁₆-C₂₅ alkyl group. In particularly preferred embodiments of Formula II, R₁₁ is a C₁₇H₃₅ group.

Another embodiment provides a compound according to Formula II wherein at least two of R₁, R₂, R₃, R₄, R₇, R₈, R₉, and R₁₀ are independently

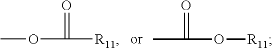

or a pharmaceutically acceptable salt or prodrug thereof, optionally in combination with an excipient.

Another embodiment provides a compound according to Formula II as described above wherein at least three of R₁, R₂, R₃, R₄, R₇, R₈, R₉, and R₁₀ are independently

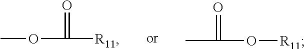

optionally in combination with an excipient.

Another embodiment provides a compound according to Formula II as described above wherein at least four of R₁, R₂, R₃, R₄, R₇, R₈, R₉, and R₁₀ are independently

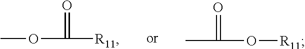

optionally in combination with an excipient.

Another embodiment provides a compound according to Formula II wherein R₁, R₂, R₃, R₄, R₇, R₈, R₉, and R₁₀ are each independently H, OH,

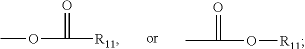

R₁₁ is a linear, branched, or cyclic, saturated or unsaturated, substituted or unsubstituted C₁-C₃₀ group, wherein if R₁₁ is cyclic, R₁₁ is a C₃-C₃₀ group;

R₅ and R₆ are independently O, —NR₁₂R₁₃ or S, wherein R₁₂ and R₁₃ are independently hydrogen, or a linear, branched, or cyclic, saturated or unsaturated, substituted or unsubstituted C₁-C₃₀ group, wherein if Ru and/or R₁₃ are cyclic, Ru and/or R₁₃ are C₃-C₃₀ groups; and

wherein at least one of R₁, R₂, R₃, R₄, R₇, R₈, R₉, and R₁₀ are independently

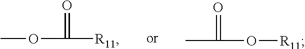

and wherein R₄ is not

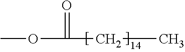

when R₁, R₂, R₃, R₇, R₈, R₉, and R₁₀ are OH;

or a pharmaceutically acceptable salt or prodrug thereof, optionally in combination with an excipient.

Another embodiment provides a composition including a compound according to Formula II wherein at least two of R₁, R₂, R₃, R₄, R₇, R₈, R₉, and R₁₀ are independently

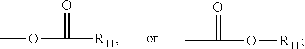

or a pharmaceutically acceptable salt or prodrug thereof, optionally in combination with an excipient.

Another embodiment provides a composition including a compound according to Formula II as described above wherein at least three of R₁, R₂, R₃, R₄, R₇, R₈, R₉, and R₁₀ are independently

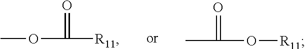

optionally in combination with an excipient.

Another embodiment provides a composition including a compound according to Formula II as described above wherein at least four of R₁, R₂, R₃, R₄, R₇, R₈, R₉, and R₁₀ are independently

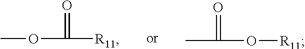

optionally in combination with an excipient.

Another embodiment provides a composition including a compound according to Formula II wherein R₁, R₂, R₃, R₄, R₇, R₈, R₉, and R₁₀ are each independently H, OH,

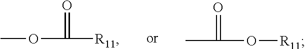

R₁₁ is a linear, branched, or cyclic, saturated or unsaturated, substituted or unsubstituted C₁-C₃₀ group, wherein if R₁₁ is cyclic, R₁₁ is a C₃-C₃₀ group;

R₅ and R₆ are independently O, —NR₁₂R₁₃ or S, wherein R₁₂ and R₁₃ are independently hydrogen, or a linear, branched, or cyclic, saturated or unsaturated, substituted or unsubstituted C₁-C₃₀ group, wherein if R₁₂ and/or R₁₃ are cyclic, R₁₂ and/or R₁₃ are C₃-C₃₀ groups; and

wherein at least one of R₁, R₂, R₃, R₄, R₇, R₈, R₉, and R₁₀ are independently

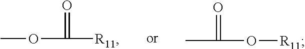

and wherein R₄ is not

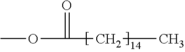

when R₁, R₂, R₃, R₇, R₈, R₉, and R₁₀ are OH; or a pharmaceutically acceptable salt or prodrug thereof, optionally in combination with an excipient.

One embodiment provides a compound according to Formula III:

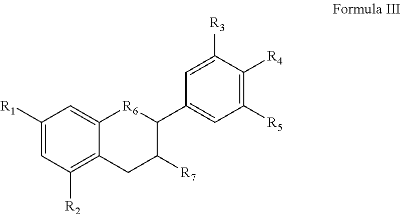

wherein R₁, R₂, R₃, R₄, R₅, and R₇ are each independently H, OH,

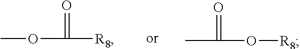

wherein R₈ is a linear or branched C₁₆-C₂₅ alkyl group.

R₆ is O, —NR₉R₁₀, or S, wherein R₉ and R₁₀ are independently hydrogen, or a linear, branched, or cyclic, saturated or unsaturated, substituted or unsubstituted C₁-C₃₀ group, wherein if R₉ and/or R₁₀ are cyclic, R₉ and/or R₁₀ are C₃-C₃₀ groups;

wherein at least one of R₁, R₂, R₃, R₄, R₅, R₇, R₉, or R₁₀ is

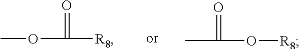

or a pharmaceutically acceptable salt or prodrug thereof, optionally in combination with an excipient.

In particularly preferred embodiments of Formula III, R₈ is a C₁₇H₃₅ group.

One embodiment provides a compound according to Formula III as described above, wherein one or more of R₁, R₂, R₃, R₄, R₅, or R₇ is

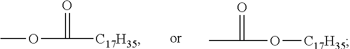

or a pharmaceutically acceptable salt or prodrug thereof, optionally in combination with an excipient.

One embodiment provides a compound according to Formula III as described above, wherein at least two of R₁, R₂, R₃, R₄, R₅, or R₇ are independently

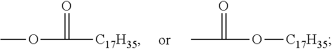

or a pharmaceutically acceptable salt or prodrug thereof, optionally in combination with an excipient.

Another embodiment provides a compound according to Formula III as described above wherein at least three of R₁, R₂, R₃, R₄, R₅, or R₇ are independently

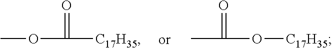

or a pharmaceutically acceptable salt or prodrug thereof, optionally in combination with an excipient.

Still another embodiment provides a compound according to Formula III as described above wherein at least four of R₁, R₂, R₃, R₄, R₅, or R₇ are independently

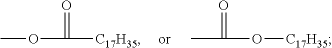

or a pharmaceutically acceptable salt or prodrug thereof, optionally in combination with an excipient.

Another embodiment provides a compound according to Formula IV:

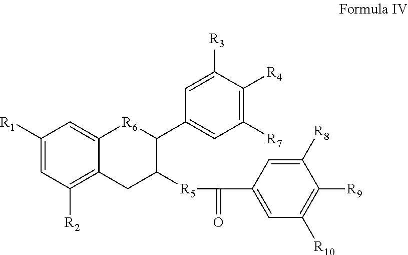

wherein R₁, R₂, R₃, R₄, R₇, R₈, R₉, and R₁₀ are each independently H, OH,

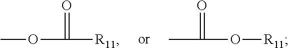

R₁₁ is a linear or branched C₁₆-C₂₅ alkyl group;

R₅ and R₆ are independently O, —NR₁₂R₁₃ or S, wherein R₁₂ and R₁₃ are independently hydrogen, or a linear, branched, or cyclic, saturated or unsaturated, substituted or unsubstituted C₁-C₃₀ group, wherein if Ru and/or R₁₃ are cyclic, Ru and/or R₁₃ are C₃-C₃₀ groups; and

wherein at least one of R₁, R₂, R₃, R₄, R₇, R₈, R₉, and R₁₀ are independently

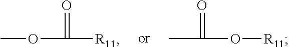

or a pharmaceutically acceptable salt or prodrug thereof, optionally in combination with an excipient.

In particularly preferred embodiments of Formula IV, R₁₁ is a C₁₇H₃₅ group.

One embodiment provides a compound according to Formula IV as described above, wherein one or more of R₁, R₂, R₃, R₄, R₇, R₈, R₉, and R₁₀ is

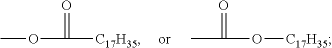

or a pharmaceutically acceptable salt or prodrug thereof, optionally in combination with an excipient.

Another embodiment provides a compound according to Formula IV wherein at least two of R₁, R₂, R₃, R₄, R₇, R₈, R₉, and R₁₀ are independently

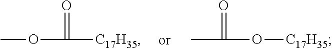

or a pharmaceutically acceptable salt or prodrug thereof, optionally in combination with an excipient.

Another embodiment provides a compound according to Formula IV as described above wherein at least three of R₁, R₂, R₃, R₄, R₇, R₈, R₉, and R₁₀ are independently

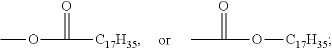

optionally in combination with an excipient.

Another embodiment provides a compound according to Formula IV as described above wherein at least four of R₁, R₂, R₃, R₄, R₇, R₈, R₉, and R₁₀ are independently

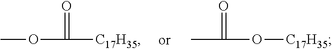

optionally in combination with an excipient.

Another embodiment provides a composition including a compound according to Formula IV wherein at least one of R₁, R₂, R₃, R₄, R₇, R₈, R₉, and R₁₀ are independently

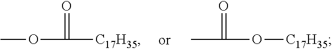

or a pharmaceutically acceptable salt or prodrug thereof, optionally in combination with an excipient.

Another embodiment provides a composition including a compound according to Formula IV wherein at least two of R₁, R₂, R₃, R₄, R₇, R₈, R₉, and R₁₀ are independently

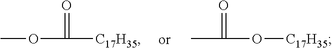

or a pharmaceutically acceptable salt or prodrug thereof, optionally in combination with an excipient.

Another embodiment provides a composition including a compound according to Formula IV as described above wherein at least three of R₁, R₂, R₃, R₄, R₇, R₈, R₉, and R₁₀ are independently

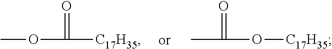

optionally in combination with an excipient.

Another embodiment provides a composition including a compound according to Formula IV as described above wherein at least four of R₁, R₂, R₃, R₄, R₇, R₈, R₉, and R₁₀ are independently

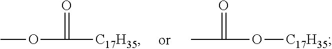

optionally in combination with an excipient.

In one embodiment, a green tea polyphenol esterified with one fatty acid is provided. Another embodiment provides a green tea polyphenol esterified with at least two fatty acids. Certain embodiments provide a green tea polyphenol esterified with one or more fatty acids having a hydrocarbon chain greater than 16 carbons. Some embodiments provide a green tea polyphenol esterified with one or more fatty acids having a hydrocarbon chain of between 17 and 25 carbons in length. Particularly preferred embodiments provide a green tea polyphenol esterified with one or more stearic acid or palmitic acid chains.

Representative green tea polyphenols include, but are not limited to (−)-epicatechin (EC), (−)-epigallocatechin (EGC), (−)-epicatechin-3-gallate (ECG), (−)-epigallocatechin-3-gallate (EGCG). Representative fatty acids include, but are not limited to butanoic acid, hexanoic acid, octanoic acid, decanoic acid, dodecanoic acid, tetradecanoic acid, hexadecanoic acid (palmitic acid), 9-hexadecenoic acid, octadecanoic acid (stearic acid), 9-octadecenoic acid, 11-octadecenoic acid, 9,12-octadecadienoic acid, 9,12,15-octadecatrienoic acid, 6,9,12-octadecatrienoic acid, eicosanoic acid, 9-eicosenoic acid, 5,8,11,14-eicosatetraenoic acid, 5,8,11,14,17-eicosapentaenoic acid, docosanoic acid, 13-docosenoic acid, 4,7,10,13,16,19-docosahexaenoic acid, and tetracosanoic acid.

1. (−)-Epigallocatechin-3-Gallate Palmitate (EGCG Palmitate) (EC16)

(−)-Epigallocatechin-3-gallate (EGCG) is the most abundant polyphenol in green tea and is a powerful antioxidant. EGCG has been shown to have numerous health benefits, including the inhibition of various human viruses. One problem with EGCG is the lack of stability of EGCG formulations. Because EGCG is an antioxidant, maintaining stability when oxygen is present is a major obstacle in producing certain formulations of EGCG. In one embodiment, the addition of acyl chains, for example via fatty acid esterification, to green tea polyphenols increases the stability of the green tea polyphenols and increases the solubility of the green tea polyphenols in hydrophobic media. The fatty acid can have C₁-C₃₀ hydrocarbon chain at one or more positions. In one embodiment, the EGCG is esterified with palmitic acid or stearic acid at the 4′ position. In a preferred embodiment, EGCG is esterified with palmitic acid at the 4′ position.

B. Methods of Esterifying Green Tea Polyphenols

Lipid esters of EGCG can be formed either enzymatically or chemically (Chen, et al., Journal of Zhejiang University Science. 2003; 6:714-718).

EGCG-ester was purified previously by Chen et al in China. This was accomplished from a catalytic esterification between green tea polyphenols and C16-fatty acid. The esterification was obtained by mixing 4 grams of green tea polyphenols and 6.5 grams of hexadecanoyl chloride. Next, 50 mL of ethyl acetate and a catalyst at 40° C. were added to the mixture. After 3 hours of stirring, the solution was washed three times with 30 mL of deionized water. The organic layer was then allowed to evaporate and further dried by using a vacuum at 40° C. This resulted in 8.7 g of powder product. A schematic of the synthesis of a likely esterification between GTP and hexadecanoyl chloride is shown below. (Chen, et al., Journal of Zhejiang University Science, 2003; 6:714-718.)

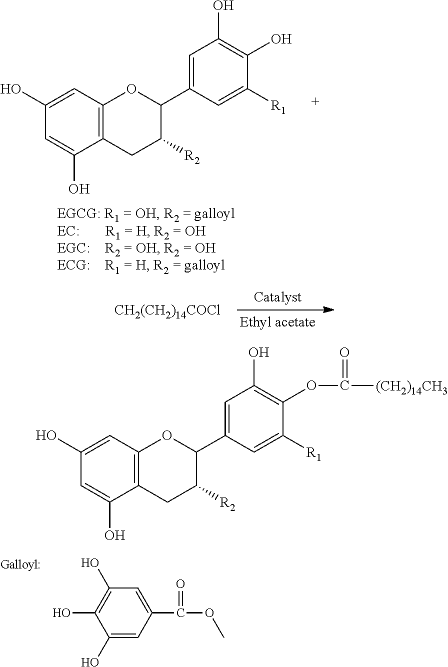

Next, high current chromatography separation was used to purify the EGCG-ester product. A two-phase solvent composed of (1:1) n-hexane-ethyl acetate-methanol-water was used in the separation column. Five grams of EGCG-ester was dissolved in 50 mL of the upper phase solution. After purification and HPLC analysis, it was seen that EGCG ester was successfully purified. The structure of an EGCG acyl-derivative is shown below. (Chen, et al., Journal of Zhejiang University Science, 2003; 6:714-718.)

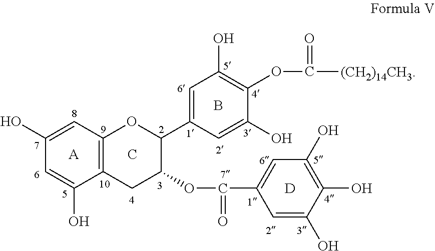

In a preferred embodiment, EGCG is esterified at the 4′ position according to the structure above with stearic acid (Formula V) or palmitic acid.

C. Pharmaceutical Compositions

Pharmaceutical compositions including the disclosed modified green tea catechins are provided. Pharmaceutical unit dosage forms of green tea catechins are suitable for oral, mucosal (e.g., nasal, sublingual, vaginal, buccal, or rectal), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs.

The composition, shape, and type of dosage forms of the green tea catechins of the disclosure will typically vary depending on their use. These and other ways in which specific dosage forms encompassed by this disclosure will vary from one another will be readily apparent to those skilled in the art. See, e.g., Remington's Pharmaceutical Sciences, 18th ed., Mack Publishing, Easton, Pa. (1990).

Another embodiment provides pharmaceutical compositions and dosage forms which include a pharmaceutically acceptable salt of one or more green tea catechins, modified green tea polyphenols, in particular, (−)-epigallocatechin-3-gallate or a pharmaceutically acceptable polymorph, solvate, hydrate, dehydrate, co-crystal, anhydrous, amorphous form thereof, and combinations thereof. Specific salts of disclosed compounds include, but are not limited to, sodium, lithium, potassium salts, and hydrates thereof.

Pharmaceutical compositions and unit dosage forms of the disclosure typically also include one or more pharmaceutically acceptable excipients or diluents. Advantages provided by specific compounds of the disclosure, such as, but not limited to, increased solubility and/or enhanced flow, purity, or stability (e.g., hygroscopicity) characteristics can make them better suited for pharmaceutical formulation and/or administration to patients than the prior art. Suitable excipients are well known to those skilled in the art of pharmacy or pharmaceutics, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets or capsules may contain excipients not suited for use in parenteral dosage forms. The suitability of a particular excipient may also depend on the specific active ingredients in the dosage form. For example, the decomposition of some active ingredients can be accelerated by some excipients such as lactose, or when exposed to water. Active ingredients that include primary or secondary amines are particularly susceptible to such accelerated decomposition.

The disclosure further encompasses pharmaceutical compositions and dosage forms that include one or more compounds that reduce the rate by which an active ingredient, for example a green tea catechin, will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate or organic acids. A specific solubility modulator is tartaric acid.

Like the amounts and types of excipients, the amounts and specific type of green tea catechin in a dosage form may depend on factors such as, but not limited to, the route by which it is to be administered to patients. However, typical dosage forms of the green tea catechin compounds of the disclosure include a pharmaceutically acceptable salt, or a pharmaceutically acceptable polymorph, solvate, hydrate, dehydrate, co-crystal, anhydrous, or amorphous form thereof, in an amount of from about 10 mg to about 1000 mg, preferably in an amount of from about 25 mg to about 750 mg, more preferably in an amount of from 50 mg to 500 mg, even more preferably in an amount of from about 30 mg to about 100 mg.

In one embodiment, the pharmaceutical compositions including the disclosed modified green tea catechins also includes a carrier, for example a sugar alcohol such as but not limited to glycerol, mannitol, sorbitol, xylitol, and erythritol. In a specific embodiment, the sugar alcohol is glycerol.

1. Formulations for Topical Administration

Topical dosage forms of disclosed modified green tea catechins include, but are not limited to, liquids, creams, lotions, ointments, gels waxes, pastes, sprays, aerosols, solutions, emulsions, and other forms know to one of skill in the art. See, e.g., Remington's Pharmaceutical Sciences, 18th ed., Mack Publishing, Easton, Pa. (1990); and Introduction to Pharmaceutical Dosage Forms, 4th ed., Lea & Febiger, Philadelphia, Pa. (1985). In a preferred embodiment, the disclosed modified green tea catechins are delivered to oral, nasal, or bronchial tissue in a suitable topical dosage form.

For non-sprayable topical dosage forms, viscous to semi-solid or solid forms including a carrier or one or more excipients compatible with topical application and having a dynamic viscosity preferably greater than water are typically employed. Suitable formulations include, without limitation, solutions, suspensions, emulsions, creams, ointments, powders, gels, waxes, pastes, liniments, salves, and the like, which are, if desired, sterilized or mixed with auxiliary agents (e.g., preservatives, stabilizers, wetting agents, buffers, or salts) for influencing various properties, such as, for example, osmotic pressure.

Nasal spray drug products contain therapeutically active ingredients dissolved or suspended in solutions or mixtures of excipients in non-pressurized dispensers that deliver a spray containing a metered dose of the active ingredient. The dose can be metered by the spray pump or could have been pre-metered during manufacture. A nasal spray unit can be designed for unit dosing or can discharge up to several hundred metered sprays of formulation containing the drug substance. Nasal sprays are applied to the nasal cavity for local and/or systemic effects.

Inhalation solution and suspension drug products are typically aqueous-based formulations that contain therapeutically active ingredients and can also contain additional excipients. Aqueous-based oral inhalation solutions and suspension must be sterile. Inhalation solutions and suspensions are intended for delivery to the lungs by oral inhalation for local and/or systemic effects and are to be used with a specified nebulizer.

An inhalation spray drug product consists of the formulation and the container closure system. The formulations are typically aqueous based and must be sterile. Inhalation sprays are intended for delivery to the lungs by oral inhalation for local and/or systemic effects. Inhalation spray drug products containing the disclosed compositions can also contain additional excipients.

Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Examples of sprayable aerosol preparations include but are not limited to metered dose inhalers, dry powder inhalers, and nebulizers. Moisturizers or humectants can also be added to pharmaceutical compositions and dosage forms if desired. Examples of such additional ingredients are well known in the art. See, e.g., Remington's Pharmaceutical Sciences, 18th Ed., Mack Publishing, Easton, Pa. (1990).

Transdermal and mucosal dosage forms of the compositions of the disclosure include, but are not limited to, ophthalmic solutions, patches, sprays, aerosols, creams, lotions, suppositories, ointments, gels, solutions, emulsions, suspensions, or other forms known to one of skill in the art. See, e.g., Remington's Pharmaceutical Sciences, 18th Ed., Mack Publishing, Easton, Pa. (1990); and Introduction to Pharmaceutical Dosage Forms, 4th Ed., Lea & Febiger, Philadelphia, Pa. (1985). Dosage forms suitable for treating mucosal tissues within the oral cavity can be formulated as mouthwashes, as oral gels, or as buccal patches. Additional transdermal dosage forms include “reservoir type” or “matrix type” patches, which can be applied to the skin and worn for a specific period of time to permit the penetration of a desired amount of active ingredient.

Examples of transdermal dosage forms and methods of administration that can be used to administer the green tea catechins of the disclosure include, but are not limited to, those disclosed in U.S. Pat. Nos. 7,097,853, 7,376,460, 7,537,590, 7,658,728, 8,386,027, 10,231,938, each of which are incorporated herein by reference in their entirety.

Suitable excipients (e.g., carriers and diluents) and other materials that can be used to provide transdermal and mucosal dosage forms encompassed by this disclosure are well known to those skilled in the pharmaceutical arts, and depend on the particular tissue or organ to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof, to form dosage forms that are non-toxic and pharmaceutically acceptable.

Depending on the specific tissue to be treated, additional components may be used prior to, in conjunction with, or subsequent to treatment with pharmaceutically acceptable salts of a green tea polyphenol of the disclosure. For example, penetration enhancers can be used to assist in delivering the active ingredients to or across the tissue. Suitable penetration enhancers include, but are not limited to: acetone; various alcohols such as ethanol, oleyl, an tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as TWEEN 80 (polysorbate 80) and SPAN 60 (sorbitan monostearate).

The pH of a pharmaceutical composition or dosage form, or of the tissue to which the pharmaceutical composition or dosage form is applied, may also be adjusted to improve delivery of the active ingredient(s). Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of the active ingredient(s) so as to improve delivery. In this regard, stearates can serve as a lipid vehicle for the formulation, as an emulsifying agent or surfactant, and as a delivery-enhancing or penetration-enhancing agent. Different hydrates, dehydrates, co-crystals, solvates, polymorphs, anhydrous, or amorphous forms of the pharmaceutically acceptable salt of a tight junction modulator can be used to further adjust the properties of the resulting composition.

The disclosed green tea catechin compositions can also be formulated as extended or delayed release formulations. Extended and delayed release formulations for various active ingredients are known in the art, for example by encapsulation.

The green tea catechin compounds, in particular the green tea catechins esterified with C₁ to C₃₀ hydrocarbon chain, are present in about 0.001% to about 50% w/v, typically from about 0.01% to about 0.1% w/v, more typically about 1% to about 20% w/v. In certain embodiments, the green tea catechins are present in about 10% w/v. In a preferred embodiment, the green tea catechin compounds are present in about 0.01% to about 20% w/v.

D. Methods of Use

The disclosed modified green tea catechin compounds and compositions thereof are useful for the treatment of one of more symptoms of viral infection. In one embodiment, the virus is a respiratory virus. The respiratory virus can be an influenza virus, respiratory syncytial virus, parainfluenza virus, adenovirus, rhinovirus, or a coronavirus. Preferably, the disclosed compositions are formulated for nasal or oral application, such as drops, applicators, or sprays. One embodiment provides green tea catechin compositions for prophylactically or therapeutically treating influenza viruses in subjects in need thereof. In one embodiment, the disclosed compositions and methods of their use thereof prevent viral infections through airborne channels.

In some embodiments, the effect of the modified green tea catechin compounds and compositions thereof on a subject is compared to a control. For example, the effect of the composition on a particular symptom, pharmacologic, or physiologic indicator can be compared to an untreated subject or the condition of the subject prior to treatment. In some embodiments, the symptom, pharmacologic, or physiologic indicator is measured in a subject prior to treatment, and again one or more times after treatment is initiated. In some embodiments, the control is a reference level, or an average determined from measuring the symptom, pharmacologic, or physiologic indicator in one or more subjects that do not have the disease or condition to be treated (for example, healthy subjects). In some embodiments, the effect of the treatment is compared to a conventional treatment that is known in the art.

1. Treating SARS-CoV-2 Infections

There is a large body of published data demonstrating EGCG or green tea polyphenols possess potent inhibitory activity against influenza virus A and B. In cell culture-based experiments, direct contact of EGCG with influenza virus results in the inactivation of viral infectivity (Song et al, Antiviral Research, 2005, Colpitts and Schang, J Virology, 2014). However, pre-incubation of cells with EGCG but no contact with the virus does not show anti-viral activity (Colpitts and Schang, J Virology, 2014). Animal studies show formulations containing EGCG can protect mice from influenza virus-induced death, only if the formulation is pre-mixed with virus (Smee et al, 2011). These data suggest that EGCG is a good candidate for preventing and treating influenza virus infection only if EGCG is in direct contact with the virus. Also, formulations with EGCG or green tea extract are not able to maintain molecular stability if oxygen is present because of the antioxidant properties of green tea polyphenols.

Methods of using the disclosed compositions to treat SARS-CoV-2 infections are disclosed herein. Methods typically include administering to the subject in need thereof an effective amount of a composition including a modified green tea catechin. In one embodiment the green tea catechin is a formulation of (−)-epigallocatechin-3-gallate palmitate (EC16) and glycerol. EC16 can be present in the pharmaceutical composition in an amount of 0.01%-0.1% w/v. In one embodiment, EC16 can be present in the pharmaceutical composition in an amount of 0.5% w/v for the treatment of SARS-CoV-2 infections. In one embodiment, the modified green tea catechin composition includes 0.01% to 0.1% w/v EC16 and 10% to 20% glycerol. In a specific embodiment, the composition includes 0.1% w/v EC16 and 20% w/v glycerol.

In one embodiment, EC16 can inhibit SARS-CoV-2 infections without negatively affecting the host cells. The disclosed modified green tea catechin compositions can be administered to a subject in need thereof nasally or orally for 1, 2, 3, 4, 5, 6, 7 days or more as needed until symptoms of SARS-CoV-2 infections have subsided. The compositions can be administered 1, 2, 3, or more times per day as needed.

2. Preventing SARS-CoV-2 Infections

Methods of using the disclosed compositions to prevent SARS-CoV-2 infections are disclosed herein. Methods typically include nasally or orally administering to the subject in need thereof an effective amount of a composition including a modified green tea catechin. In one embodiment the green tea catechin is a formulation of (−)-epigallocatechin-3-gallate palmitate (EC16) and glycerol. Without being bound to any one theory, it is believed that the disclosed modified green tea catechins protect airway epithelial cells of the nose, mouth, and lung from viral infection. Prophylactic administration of the disclosed modified green tea catechin formulations to subjects who are not actively infected with SARS-CoV-2 can prevent them from becoming infected with SARS-CoV-2. EC16 can have a “coating” effect on the airway epithelial cells, possibly through the insertion of the fatty acyl chain into the hydrophobic portion of cell membrane, thereby retaining EGCG to inactivate virus when the virus subsequently encounters the cell membrane. Another potential mechanism is the binding of EC16 to cell surface sialic acid-containing glycoproteins, thereby preventing the binding and internalization of H1N1 into the cells.

EC16 can be present in the pharmaceutical composition in an amount of 0.01%-0.1% w/v. In another embodiment EC16 is present in the pharmaceutical composition in an amount of 0.2% w/v. In one embodiment, the modified green tea catechin composition includes 0.01% to 0.1% w/v EC16 and 10% to 20% glycerol. In a specific embodiment, the composition includes 0.1% w/v EC16 and 20% w/v glycerol. The disclosed compositions can be administered prophylactically three times daily, twice daily, once daily, or every other day. In a preferred embodiment, the disclosed compositions are administered twice daily. In one embodiment, the modified green tea catechin composition is administered to the subject continuously during times of high influenza activity. In another embodiment, subjects are administered the disclosed compositions on a daily basis for an extended period of time, for example six-months, one-year, two-years, or more than two years.

a. High Risk Subjects

In some embodiments, certain subjects are at a higher risk of being infected with influenza virus. Subjects that are at high risk for developing SARS-CoV-2 infections but are not limited to people 65 years and older, pregnant women, young children, and people with certain chronic medical conditions such as asthma, diabetes, or heart disease. These high risk subjects are not only more likely to become infected with the flu, but are also more likely to suffer from complications from the flu. Examples of flu related complications include but are not limited to pneumonia, bronchitis, sinus infections, and ear infections. Complications can result in hospitalization, and in some cases, death. In one embodiment, high risk subjects can be administered the disclosed compositions to prophylactically treat SARS-CoV-2 infections.

In one embodiment, high risk subjects are continuously administered the disclosed modified green tea catechin compositions during times of high influenza activity. The high risk subjects can be administered the disclosed compositions 1, 2, 3, or more times daily.

3. Other Respiratory Viruses

Methods of using the disclosed compositions to prevent respiratory viral infection are disclosed herein. Methods typically include nasally or orally administering to the subject in need thereof an effective amount of a composition including a modified green tea catechin to inhibit or reduce entry of the virus into respiratory epithelial cells of the subject. In one embodiment, the disclosed compositions at least partially coat the nasal or respiratory cells to inhibit or reduce entry of the virus into respiratory epithelial cells. In one embodiment the green tea catechin is a formulation of (−)-epigallocatechin-3-gallate palmitate (EC16) and glycerol. Exemplary respiratory viruses include but are not limited to respiratory syncytial virus, parainfluenza virus, adenovirus, rhinovirus, and coronavirus. In one embodiment, the coronavirus is SARS-CoV-2. (−)-epigallocatechin-3-gallate (EGCG)

### EXAMPLES

### Example 1: EGCG Reduces SARS-CoV-2 Yield in Caco-2 Cells and Blocks SARS-CoV-2 Infection without Apparent Cytotoxicity (Reference: Hurst et al, Epigallocatechin-3-Gallate (E:GCG) Inhibits SARS-CoV-2 Infection in Primate Epithelial Cells. Microbiology & Infectious Diseases, 2021)

The Primary CPE assay results measuring EC50 in Vero cells was performed. The results shows that EGCG inhibited CPE with EC50 of 0.23 (VIS) to 0.27 (NR) μg/ml, while the CC50 was 3.2 (VIS) to 2.3 (NR) μg/ml. The secondary VYR assay was conducted with human epithelial cells to validate the antiviral activity with EC90 values.

Methods

Cells, Virus and Compound

The human epithelial cell line Caco-2 was used. SARS-CoV-2 virus and epigallocatechin-3-gallate (EGCG, water-soluble form) (CAS number 989-51-5) compound were used.

The secondary VYR assay in Caco-2 cells: Near-confluent cell culture monolayers of Caco-2 cells were prepared in 96-well disposable microplates the day before testing. Cells were maintained in MEM supplemented with 10% FBS. The same medium was used for antiviral assays and FBS was reduced to 2%, supplemented with 50-μg/ml gentamicin. EGCG was dissolved in serum-free MEM in a series of 2×dilutions from 100 μg/ml to 0.78 μg/ml. The EGCG diliutions were mixed and incubated with SARS-CoV-2 at 200 CCID₅₀ (50% cell culture infectious dose) in 0.1 ml volume for 1 h prior to adding to the cell culture for 1 h absorption. Following absorption, the unabsorbed SARS-CoV-2 along with medium/EGCG was removed and fresh MEM was added to the cell culture for viral yield detection in comparison to untreated infection control. Five microwells were used per dilution: three for infected cultures and two for uninfected toxicity cultures. Controls for the experiment consist of six microwells that were infected and not treated (virus controls) and six that were untreated and uninfected (cell controls) on every plate. Medium devoid of virus was placed in toxicity control wells and cell control wells. Plates were incubated at 37° C. with 5% CO2 for 72 hours. A sample of supernatant was taken from each infected well (three replicate wells were pooled) and tested immediately. To evaluate toxicity, the plates were stained with 0.011% neutral red dye for approximately two hours at 37° C. in a 5% CO2 incubator. The neutral red medium was removed by complete aspiration, and the cells were rinsed 1× with phosphate buffered solution (PBS) to remove residual dye. The PBS is completely removed, and the incorporated neutral red was eluted with 50% Sorensen's citrate buffer/50% ethanol for at least 30 minutes. As larger the number of viable cells present in the wells, when the neutral red dye penetrated into the living cells, the more intense the red color appeared. The dye content in each well was quantified using a spectrophotometer at 540 nm wavelength. The dye content in each set of wells was converted to a percentage of dye present in untreated control wells using a Microsoft Excel computer-based spreadsheet and normalized based on the virus control. The 50% cytotoxic (CC₅₀, cell-inhibitory) concentrations were then calculated by regression analysis.

The VYR test indicates how much EGCG inhibits virus entry and replication. Virus yielded in the presence of EGCG was titrated and compared to virus titers from the untreated virus controls. Titration of the viral samples (collected as described in the paragraph above) was performed by endpoint dilution (Reed-Muench method). Serial 1/10 dilutions of virus were made and plated into 4 replicate wells containing fresh cell monolayers of Vero 76 cells. Plates were then incubated, and cells were scored for the presence or absence of virus after distinct CPE was observed. The TCID₅₀ values were calculated using the Reed-Muench method. The 90% (one log₁₀) effective concentration (EC₉₀) was calculated by regression analysis by plotting the log₁₀ of the inhibitor concentration versus log₁₀ of virus produced at each concentration. The quotient of CC₅₀ divided by EC₉₀ gave the selectivity index (SI) value. Compounds showing SI values ≥10 are considered active.

Results

In-vitro antiviral (SARS-CoV-2) screening Report: The in-vitro antiviral (SARS-CoV-2) screening test using EGCG was performed in NIAID-designated laboratory at Utah State University. The in-vitro antiviral (SARS-CoV-2) screening test using EGCG demonstrated that while the cytotoxicity was minimal (CC50 >100 μg/ml), EGCG inhibited 90% of viral yield at 28 μg/ml (0.0028%) without CPE (cytopathic effect). Thus, EGCG blocked SARS-CoV-2 infection effectively without aparent cytotoxicity. This result is consistant with our previously published data on influenza virus, H1N1, another respiratory virus.

The results in Table 1 demonstrated that at 50 μg/ml (0.005%), EGCG caused 1.8 log 10 reduction in virus titer, which translates to 98.41% reduction of viral yield. Further, at 100 μg/ml (0.01%), EGCG resulted in >3.6 log 10 reduction in virus titer, which translates to >99.975% reduction of viral yield. While when EGCG concentration was at or lower than 25 μg/ml (0.0025%), the activity was not detected.

TABLE 1
Virus titers of
EGCG from Caco 2 cells
infected with SARS-CoV-2.

| | Viral |
|---|---|
| | titer of |
| | EGCG |
| | treated |
| | TCID50 |
| Concentration | values |
| (μg/ml) | lon₁₀ |
| 100 | <1.7 |
| 50 | 3.5 |
| 25 | 5.0 |
| 12.5 | 5.3 |
| 6.25 | 5.5 |
| 3.13 | 5.0 |
| 1.56 | 5.0 |
| 0.78 | 5.3 |

Visual (virus yield reduction)/Neutral Red (Toxicity) assay using compound, EGCG and virus, SARS-CoV-2 was performed.

EC₉₀ is 4.3; CC₅₀ is >100 and SIgo is >23.

EC₅₀—compound concentration that reduces viral replication by 50%

EC₉₀—compound concentration that reduces viral replication by 90%

CC₅₀—compound concentration that reduces cell viability by 50%

SIgo—CC₅₀/EC₅₀

SIgo—CC₅₀/EC₉₀

Compounds with SI values >10 are considered active.

### Example 2: EGCG, GCG and EGCG-Palmitate Inhibits SARS-CoV Infection and SARS-CoV Replication

### Methods

### Compounds, Virus and Host Cell Target:

Compounds such as epigallocatechin-3-gallate (EGCG, water-soluble form) (CAS number 989-51-5), EGCG-palmitate (lipid soluble form) (CAS number 144948-10-7) and gallocatechin gallate (GCG) (epimer of EGCG were purchased from Camellix LLC. SARS-CoV virus and transmembrane protease serine 2 (TMPRSS2) as host cell target were used.

Currently, there is no in vivo data for EGCG on SARS-CoV-2. There are several in vitro studies suggest that EGCG is a potential candidate as an inhibitor of viral entry and replication. Transmembrane protease serine 2 (TMPRSS2) is an enzyme in human that is encoded by TMPRSS2 gene. TMPRSS2 is a cell surface protein primarily expressed by endothelial cells across the respiratory and digestive tracts. Certain viruses, especially coronaviruses including SARS-CoV (that causes SARS), MERS-CoV (causes MERS), and SARS-CoV-2 (which causes COVID19), require TMPRSS2 for their entry into the body.

Results

An in vitro study showed that EGCG inhibited TMPRSS2, one of the two viral entry proteins and the key transmembrane serine protease for SARS-Co-V-2 S protein priming (S1, S2). Inhibition of TMPRSS2 would interrupt the entry of SARS-CoV-2 into host cells.

Viral target 1: Nucleocapsid protein (N protein). In an in vitro screening of polyphenolic compounds, gallocatechin gallate (GCG) (epimer of EGCG) exhibited remarkable inhibitory effect against SARS-CoV nucleocapsid (N) protein, which could be an antiviral target. N protein played major role in the binding of viral RNA genome and packing the long helical nucleocapsid complex. Other important functions of the N protein are induction of host cell apoptosis, modulation of host-pathogen interaction and immune response (S3, S4).

Viral target 2: Chymotrypsin-like cysteine protease, or 3C-like protease (3CL^{Pro}). Another potential drug target, 3C-like protease, play a vital role for SARS-CoV replication. EGCG and GCG significantly inhibited SARS-CoV replication 3C-like protease, leading to interruption of viral replication. The proteolytic cleavage of viral polyproteins by SARS-CoV 3CL^{pro} is the major control point of SARS-CoV viral replication, and EGCG inhibited 80% 3C^{pro} activity (S5, S6).

### Example 3: Therapeutic and Prophylactic Use of EGCG and EGCG Palmitate

Methods

Compounds such as epigallocatechin-3-gallate (EGCG, water-soluble form) and EGCG-palmitate (lipid soluble form or EC16) were purchased from Camellix, LLC.

EGCG is stable in dry powder form, but not stable in aqueous solution. Powder form is stable at 5° C. for two years. EGCG-palmitate is stable in dry powder form, and in proper liquid formulations. Powder form is stable at 5° C. for two years.

Results

Formulation

EGCG is suitable for oral administration as tablets or capsules.

EGCG-palmitate is suitable for liquid nasal applications, as well as for oral administration in the form of tablets or capsules.

### Example 4: EC16 Inactivates H1N1 Virus

Methods

Cells and Virus: MDCK cells were purchased from ATCC and cultured in MEM cell culture medium supplemented with 10% fetal calf serum and tri-antibiotics. H1N1 virus was purchased from ATCC and stored at −80° C.

Infection of MDCK cells and TCID50 viral titer assay: MDCK cells were cultured in Minimum Essential Media (MEM, Life Technologies Corporation, Carlsbad, Calif.) supplemented with 10% fetal calf serum (Heat inactivated, Neuromics, Edina, Minn.) and 1× penicillin, streptomycin, and amphotericin B (Corning, Corning, N.Y.). The viral infection assay was performed in 96 well cell culture plates (tissue culture treated, Southern Labware, Cumming, Ga.) using MDCK cells that had reached confluency. A series dilution of H1N1 virus stock to 10⁻⁷ fold was prepared using MEM serum-free medium with antibiotics, and 100 μl of each viral mix dilution was loaded into wells with four replicate wells per dilution. After a one hour incubation, the viral dilutions were removed and 200 μl MEM serum-free medium with 0.2 μg/ml trypsin (Life Technologies Corporation, Carlsbad, Calif.) was added to the wells, followed by incubation at 35° C. with 5% CO2 for 4 days to allow a CPE (Cytopathic effect) to become visible. According to the TCID50 protocol and software (Reed, et al., Am J Epidemiol, 27:493-497 (1938)), the number of wells showing CPE was entered into the calculation formula to determine the infection activity of the virus (titer). The viral titer without any EC16 treatment was set as 100%. The remaining viral infection titer from various EC16 treatments was determined and the percentage of the untreated infection rate was calculated.

Viral inactivation test: Formulations containing EC16 were made by dissolving EC16 in glycerol (referred to as “carrier” hereafter) and then diluting with a mixture of MEM serum free medium and carrier to 0.01% (w/v) or 0.1% (w/v) EC16 in 10% or 20% carrier. In a 2 ml micro centrifuge tube, 50 μl of H1N1 virus stock was added to 450 μl of a formulation containing EC16 and carrier. The tube was then closed and the contents mixed by shaking for 60 sec of direct contact, and the viral/EC16 mix was then immediately diluted 10× in MEM serum-free medium (100 μl mix to 900 μl MEM) in order to inactivate EC16, followed by series 10-fold dilutions to 10-6. The dilutions were loaded onto MDCK cell monolayers in a 96 well plate (100 μl per well, 4 repeats). After 1 hour absorption, the dilutions were removed, and cells incubated and the viral infection rate determined as described above.

Results

Pilot experiments showed no antiviral activity for 10% or 20% carrier alone in MEM serum-free medium (data not shown; p>0.4).

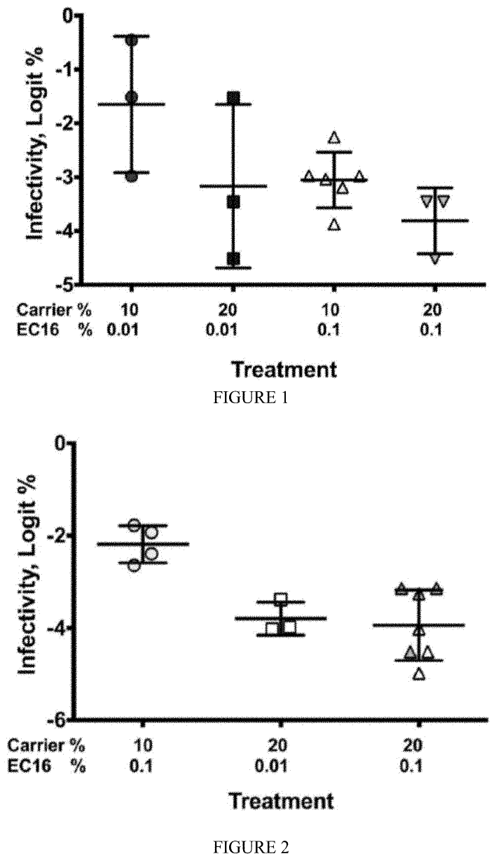

This experiment was designed to determine whether EC16 was capable of inactivating H1N1 virus rapidly by direct contact for 1 minute after mixing with the virus. FIG. 1 shows that EC16 at 0.01% in a formulation containing 10% carrier reduced H1N1 infectivity to 20.5%±17.1 of control, (n=3) while 0.1% EC16 reduced infectivity to 6.1%±3.0. For 0.1% EC16 in 20% carrier, the value was 2.4%±1.1. Matching was not effective (p=0.6; n=3, logit transformed values). An additional three separate replicate experiments testing 0.1% EC16 in 10% carrier showed consistent results (3.8%±1.5; overall mean (n=6) 5.0%±2.4 of control), and in separate experiments testing 0.01% EC16 in 20% carrier, infectivity was reduced to 7.3%±9.2% of control.

Ordinary two-way ANOVA using all values showed no significant interaction (p=0.48), and no significant effect for EC16 concentration (p=0.07), but a borderline significant effect for carrier concentration (p=0.048). (However, with the different group sizes and low n, these p values should be viewed with caution.) The main trend was thus for a reduced standard deviation at 0.1% EC16, suggesting a more consistent treatment effect. All four test groups gave significantly less than 99.9% viral activity (one-sample t-test, p≤0.008), and all but 0.01% EC16, 20% carrier (p=0.020; not significant after Bonferroni correction n=4) were significantly higher than 0.01% viral activity (p<0.004). That is, the reduction in activity was significant, but broadly, it remained significantly above 0% viral activity.

### Example 5: EC16 Prevents H1N1 Infection

Methods

Prevention test: Different EC16 formulations (100 μl) were incubated with MDCK cells for 1 hour in a cell culture incubator, followed by formulation removal and washing with MEM serum-free medium. A serial dilution of H1N1 virus in MEM serum-free medium was added to confluent monolayers of MDCK cells, and incubated for 1 hour. As described above, the medium was changed and TCID50 infection rate determined after 4 days of incubation.

Results

To test the ability of EC16 to prevent cell infection by H1N1, MDCK cell monolayers were incubated with EC16 treatments for 1 hour, and then free EC16 was washed away before cells were exposed to virus. Two sets of experiments were performed: the first compared the effects of 0.01% EC16 in 10% carrier, 0.1% EC16 in 10% carrier, and 0.1% EC16 in 20% carrier (n=4); the second compared 0.01% and 0.1% EC16 in 20% carrier (n=3). At 10% carrier, 0.01% EC16 showed no effect on viral titer (100% viability, n=4; data not shown). There was no significant matching effect in either experiment (p≥0.34), and the mean viral titers from the two sets of experiments using 0.1% EC16 20% carrier also did not differ significantly (unpaired t-test; p=0.08). Therefore, results from the two experiments were combined for analysis (FIG. 2). The values (n=7) for 0.1% EC16, 20% carrier were normally distributed (Shapiro-Wilk test, p=0.06).

All three of these treatments (0.1% EC16 in 10 and 20% carrier, 0.01% EC16 in 20% carrier) gave infectivity values significantly less than 99.9% of control (p<0.004), but greater than 0.01% (p<0.002); the means (±SD) for 0.1% EC16, 10% carrier, and 0.01 and 0.1% EC16 in 20% carrier were respectively 10.5%±3.6, 2.3%±0.9, and 2.4%±1.6. Ordinary one-way ANOVA of logit transformed data showed a significant difference between the groups (p=0.003), with the mean for 0.1% EC16, 10% carrier being significantly higher compared to the other groups (p<0.015), consistent with the effect of carrier concentration seen in the suspension test. The standard deviations did not differ significantly (Brown-Forsythe test, p=0.11).

To further examine the role of carrier in the EC16 reduction of viral titer, we used 2% dimethyl sulfoxide (DMSO) as an EC16 solvent for the prevention test with different concentrations of EC16 instead of carrier. EC16 at concentrations from 0 to 0.1% in DMSO did not result in a statistically significant difference in comparison to the control viral titer (data not shown). Carrier content in the formulation is required for the reduction in viral titer because the identical concentration of EC16 with 2% DMSO failed to deliver similar outcomes.

Collectively, these results showed that EC16 in the presence of carrier could substantially reduce MDCK infection by H1N1 virus, and that 0.1% EC16 in 20% carrier gave the greatest (42-fold) reduction in titer.

To test if EC16 formulation has a long-lasting preventive effect, cells were pre-treated with EC16 formula for 1 hour, and the formula was washed away. New medium without EC16 was added to the cell culture and incubated for 1 hour. Then the cells were infected with H1N1 virus for 1 hour. This experiment allowed the EC16 coating on cells to be washed away and left the cells without EC16 exposure for one hour before the cells were infected with H1N1 virus for one hour.

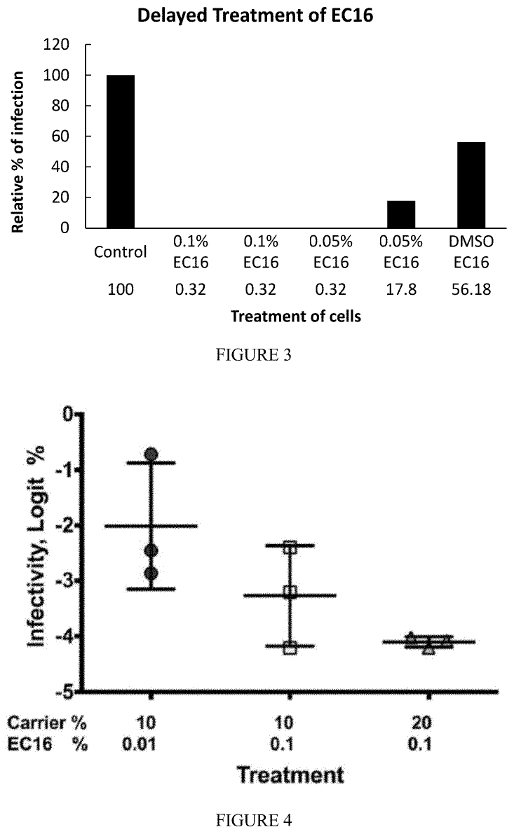

The results demonstrated that 0.1% EC16 almost completely blocked the H1N1 infection 2 hours after the first application of the formula, even when the EC16 was washed away and cells were incubated without EC16 for one hour (FIG. 3). At 0.05% EC16, an average of 90% of cells were protected from H1N1 infection.

### Example 6: EC16 Treats H1N1 Infection

Methods

Treatment test: To test if formulations containing EC16 had a treatment (post-infection) effect, MDCK cells in 96 well cell culture plate were initially infected for one hour with H1N1 virus in series dilutions. Then, 100 μl of formulations containing EC16 were applied to each well onto the cells for one hour before being washed away with MEM serum-free medium. The TCID50 was determined as described above.

Results

To determine if EC16 was capable of reducing viral reproduction in MDCK cells that had just been infected with virus, monolayers of MDCK cells were infected with a series of dilutions of H1N1 for 1 hour, then EC16 was applied at either 0.01% in 10% carrier, or 0.01 or 0.1% in 20% carrier. All treatment values were significantly lower than controls (99.99%, p<0.005), indicating an antiviral effect, but significantly higher than 0.01% of control (p<0.008), consistent with some remaining active virus. FIG. 4 demonstrates that 0.01% EC16 in 10% carrier was relatively poor and somewhat inconsistent at treating infected cells (viral TCID50 reduced to 15.4%±15.2 of control).

However, at 0.1%, EC16 in 10% or 20% carrier reduced the viral titer to respectively 4.6%±3.5 and 1.6%±0.2 of control. Matching was not significant (p=0.3), and the standard deviations did not differ significantly (Brown-Forsythe test, p=0.42). Analysis of the three groups by ordinary one-way ANOVA showed no significant difference between the three treatments (p=0.057; n=3).

### Example 7: Thin Layer Coating Test

Methods

Thin Layer Coating Test: To test if a thin layer of formulations containing EC16 applied on top of a cell monolayer could reduce H1N1 infection, 10 μl of formulations containing EC16 was applied to each well (0.3 cm2 in area) of a 96-well plate of MDCK cells for either 10 or 30 min. Then, the cells were exposed for 1 hour to an H1N1 challenge in series dilutions without removal of the formulation. The viral dilutions were removed and 200 μl fresh MEM serum-free medium with 0.2 μg/ml trypsin added, and TCID50 determined as above.

Results

These experiments were designed to determine whether a thin layer of EC16 formulation coating the monolayer of MDCK cells (33 μl/cm2 well areas) prevented subsequent H1N1 viral infection. In the first set of experiments, cells were treated for 10 minutes with 0.01 and 0.1% EC16 in 10% carrier, and 0.1% EC16 in 20% carrier for 10 and 30 minutes. In the second set of experiments, 0.01 and 0.1% EC16 in 20% carrier were compared at 30 minutes of treatment (FIG. 5). Matching was not effective (p≥0.4) and there was no significant difference between the two sets of data for a 30 minute treatment with 0.1% EC16 in 20% carrier (unpaired t-test, p=0.94). Therefore, the two sets of data were combined for analysis.

EC16 at 0.01% with 10% carrier for 10 minutes gave inconsistent and poor viral inhibition (remaining viability 48.4%±46.4), with remaining infectivity ranging from 10% to 100%. This group was therefore excluded from subsequent analysis. The 10 minute treatment groups with 0.1% EC16 in 10% and 20% carrier, and the 30 minute treatment groups with 0.01% and 0.1% EC16 in 20% carrier, gave mean values of respectively 9.5%±1.4, 12.1%±5.8, 7.6%±7.2, and 0.9%±0.7. These were all significantly less than 99.9% (one-sample t-test, p≤0.007), but (with the exception of 0.01% EC16, 20% carrier, 30 min, p=0.016, not significant after Bonferroni correction, n=4), significantly greater than 0.01% (p≤0.003). For the two 10 minute 0.1% EC16 treatments, there was no significant difference in viral titer between 10% and 20% carrier (unpaired t-test with Welch's correction, p=0.65).

Similarly, there was no significant difference between 0.01% and 0.1% EC16 with a 30 minute treatment in 20% carrier. However, when 10 minute versus 30 minute treatments with 0.1% EC16 in 20% carrier were compared, a 30-minute treatment gave a significantly greater reduction in titer (p=0.004). Similar results were obtained analyzing the experiments separately.

### Example 8: Cell Viability with EC16 Treatment

Methods:

Cell Viability: This experiment tested if EC16 formulations were associated with cytotoxicity in MDCK cells. MDCK cells were cultured in a 96 well plate until confluent. MEM serum free medium, MEM medium with 10% carrier and 20% carrier (carrier controls), or MEM medium containing 0.1% EC16 and 10% or 20% carrier was added to the wells followed by a 1 hour incubation at 35° C. with 5% CO2. The medium was then changed to 200 μl fresh MEM serum free medium with 0.2 μg/ml trypsin in each well and incubated overnight under the same condition. The plate was removed from the cell culture incubator and an MTT assay was performed according to a method described previously (Yamamoto, T., et al., Anticancer Research, 24:3065-3073 (2004)).

Results:

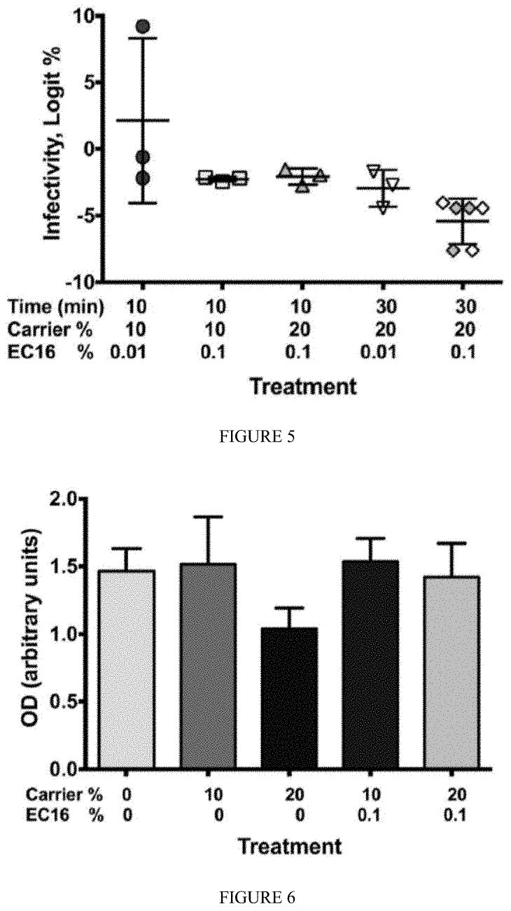

This experiment was designed to determine if EC16 induced cytotoxicity in MDCK cells. Repeat measures one-way ANOVA showed a significant effect in treatment groups (p<0.0001; matching was effective (p=0.033); Geisser-Greenhouse epsilon 0.638). As shown in FIG. 6, there was a significant decrease in cell viability induced by 1 hour incubation with 20% carrier alone in comparison to all four treatments (p<0.0005; a 29% reduction in MTT value in comparison to MEM alone, 1.04±0.15 vs. 1.47±0.17 OD units). However, 10% carrier, and EC16 containing formulations with either 10% or 20% carrier, were not statistically different from the MEM control (p>0.6). That is, EC16 protected the cells from the cell viability reduction (or a reduction in metabolic rate as determined by the MTT assay) associated with a high carrier concentration.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for inhibiting or reducing respiratory viral infection in a subject comprising:
administering to the subject an effective amount of a composition comprising at least one green tea polyphenol esterified with a C₁-C₃₀ group in at least one position and a carrier to inhibit or reduce entry of SARS-CoV-2 into respiratory epithelial cells of the subject.

2. The method of claim 1, wherein the carrier is either in water soluble form or in lipid soluble form.

3. The method of claim 2, wherein the carrier is water.

4. The method of claim 2, wherein the carrier is glycerol.

5. The method of claim 1, wherein the green tea polyphenol is selected from the group consisting of (−)-epicatechin, (−)-epigallocatechin, (−)-epicatechin-3-gallate, and proanthocyanidins.

6. The method of claim 1, wherein the modified green tea polyphenol is (−)-epigallocatechin-3-gallate (EGCG).

7. The method of claim 1, wherein the modified green tea polyphenol is (−)-epigallocatechin-3-gallate-palmitate (EGCG-palmitate or EC16).

8. The method of claim 1 wherein the composition is administered by bronchial, pulmonary, nasal or oral administration.

9. The method of claim 1, wherein the respiratory epithelial cells comprise ciliated cells, goblet cells, basal cells, epidermal cells and combinations thereof.

10. The method of claim 1, wherein the respiratory epithelial cells comprise nasal epithelial cells, oral epithelial cells, bronchial epithelial cells, or combinations thereof.

11. The method of claim 1, wherein the respiratory epithelial cells are at least partially coated with the composition.

12. The method of claim 1, wherein the composition comprises 0.01%-20% w/v of the esterified green tea polyphenol.

13. The method of claim 1, wherein the respiratory virus is selected from the group consisting of an influenza virus, respiratory syncytial virus, parainfluenza virus, adenovirus, rhinovirus, and coronavirus.

14. The method of claim 13, wherein the coronavirus is SARS-CoV-2.

15. A method of reducing the risk of a viral infection in a subject comprising administering to the subject an effective amount of a prophylactic composition comprising (−)-epigallocatechin-3-gallate-palmitate and glycerol to inhibit or reduce viral infection in respiratory epithelial cells of the subject.

16. The method of claim 15, wherein the composition comprises 0.1% w/v (−)-epigallocatechin-3-gallate-palmitate and 20% w/v glycerol.

17. The method of claim 15, wherein the composition is formulated for nasal, oral, pulmonary, or bronchial administration.

18. The method of claim 15, wherein the viral infection is SARS-CoV-2 infection.

19. A method for preventing the replication of respiratory viral infection in a subject comprising:
administering to the subject an effective amount of a composition comprising at least one green tea polyphenol esterified with a C₁-C₃₀ group in at least one position and a carrier to prevent entry of SARS-CoV-2 into respiratory epithelial cells of the subject.

20. A composition comprising:
a prophylactically effective amount of epigallocatechin-3-gallate-palmitate and glycerol to inhibit or reduce viral entry into respiratory epithelial cells of a subject, wherein the composition is formulated for nasal, bronchial, or pulmonary administration.

21. The composition of claim 20, wherein the composition is an aerosol formulation.

22. The composition of claim 21, wherein the composition is a liquid aerosol or a powdered aerosol.

23. The composition of claim 20, wherein the composition is formulated as a topical formulation.

24. The composition of claim 20, wherein the composition is a liquid, gel, wax, vaper or paste.

25. The composition of claim 20, wherein the virus is a respiratory virus.

26. The composition of claim 20, wherein the virus is selected from the group consisting of an influenza virus, respiratory syncytial virus, parainfluenza virus, adenovirus, rhinovirus, and coronavirus.

27. The composition of claim 26, wherein the coronavirus is SARS-CoV-2.
